(19) United States
(12) Patent Application Publication
(10) Pub. No.: US 2021/0353180 A1
(43) Pub. Date: Nov. 18, 2021

(54) SYSTEM AND METHOD FOR SMART DENTAL UNIT

(71) Applicant: Cydent LLC, Greenwood Village, CO (US)

(72) Inventors: Lynn Bolton Fischer, Centennial, CO (US); Charles William Fischer, III, Centennial, CO (US); Melissa Fischer Alconcel, Aurora, CO (US)

(73) Assignee: Cydent LLC, Greenwood Village, CO (US)

(21) Appl. No.: 17/307,085

(22) Filed: May 4, 2021

Related U.S. Application Data
(60) Provisional application No. 63/025,032, filed on May 14, 2020.

(51) Int. Cl. A61B 5/11; A61C 13/01; A61B 5/00; A61B 5/1455; A61B 5/01; A61C 13/00; A61C 8/00

(52) U.S. Cl. CPC A61B 5/1117; A61C 13/01; A61B 5/0002; A61B 5/682; A61B 5/14552; A61B 5/01; H02J 7/02; A61C 13/0019; A61C 8/0095; A61B 2562/0219; A61B 2562/0271; A61B 2560/0214; A61B 2562/08; A61B 5/7405

(57) ABSTRACT

Provided is a system and method for smart dental unit. The system provides smart dental unit restoring one or more teeth, including: a prosthetic base structured and arranged to engage with mouth tissue. The prosthetic base supports at least one tooth and at least one sensor system structured and arranged to detect at least one event. The sensor system further has a processor coupled to a wireless transceiver operable to communicate with at least one remote computing system. The smart dental unit also has a unique identifier. In response to a detected event the processor generates data communicated by the wireless transceiver to the remote computer. The unique identifier is also used by the remote computer to determine the location of the smart dental unit. An associated method for making a smart dental unit and a system for dental based patient care are also disclosed.

### CROSS REFERENCE TO RELATED APPLICATIONS

This application claims the benefit under 34 U.S.C. § 119(e) of U.S. Provisional Application No. 63/025,032 filed May 14, 2020 and entitled SYSTEM AND METHOD FOR KINEMATIC-SENSING AND LOCATION MONITORING OF DENTAL PROSTHESES AND APPLIANCES, the disclosure of which is incorporated herein by reference.

In addition, the present application also incorporates by references herein U.S. Provisional Application No. 63/112,276 filed Nov. 4, 2020 and entitled SYSTEM AND METHOD FOR KINEMATIC-SENSING, TEMPERATURE, AND LOCATION MONITORING OF DENTAL PROSTHESES AND APPLIANCES, the disclosure of which is incorporated herein by reference.

### FIELD OF THE INVENTION

The present invention relates generally to dental prosthesis devices having embedded technology. Such technology relates to kinematic sensing, temperature, and location monitoring. Having kinematic and temperature sensing capabilities in dental appliances provides the capability to assess the moving and temperature state of both the appliance and the person wearing the dental appliance. Such a dental device can be leveraged in multiple ways including, but not limited to, dental appliance loss prevention, dental treatment compliance, body temperature trending, and the assessment of human falls whether the person is conscious or unconscious, and whether the impact injury is trivial or traumatic.

### BACKGROUND

Considerable advances in sensors, including location technology (e.g. RFID, BLE, and GPS), temperature, and motion detection (e.g. accelerometers) and subsequent miniaturization provides the opportunity to leverage such technology in the dental domain.

More than 36 million Americans do not have any teeth or are missing one arch (edentulism). In all, 122 million people in the U.S. are missing at least one tooth, not including wisdom teeth. 90% of those who suffer from edentulism have dentures. About 14% of the edentulous population has dentures made each year. The number of edentulous individuals will continue to increase in the next 14 years to more than 200 million individuals, Ref. American College of Prosthodontics.

The cost of dental devices and appliances is significant. For example, Long-Term Care Facilities (LTCs) have significant loss of devices. With a rough estimate of 2 to 4 lost appliances per month per LTC facility, this can lead to $48K to $96K loss per year for a facility. For a midsize LTC company of 100 facilities, this would be a yearly loss potentially nearing $10M. The cost of replacement is incurred by the LTCs, the device owners, and insurance providers (Medicare replaces every 7 years). In many cases the families of the LTC residents bear the burden of the costs.

Compliance with prescribed dental appliance treatment is a challenge for the majority of denture owners. Non-compliance for people with dementia is 100% and requires care provider support. High levels of non-compliance result in lack of removal for very long periods of time or failure to use the appliance as often as needed. This non-compliance results in poor oral hygiene and can lead to systemic problems such as increased risk of infection, more likelihood of developing ulcers, untreated open wounds and eating issues. Responsibility for dental compliance may be with the individual, family, or long-term care providers. A compliance monitor exists for orthodontic appliances; however, the monitor focuses on cumulative-time compliance data related to the desired position of the teeth.

In addition, it is a well-recognized fact that elderly people tend to suffer greater injury from an accidental slip or fall due to the general issues of aging—e.g., loss of muscle mass, decrease in bone density, the body's ability to regenerate and heal, etc. Notification systems have been developed to aid a fallen person to call for aid should the party suffer a fall. However, in the vast majority of cases, the fallen person must be conscious and able to activate the device and/or able to crawl to it—such as in the case of a pull line. For wearable alert systems, the person must remember to put it on or otherwise take it with them.

Additionally, most of these devices do not measure the magnitude of a fall. This lack of information can lead to issues as the devices may not properly distinguish between a person “dropping” themselves into a chair, or truly falling. Such ambiguity may see the dispatch of aids when none are required, and no dispatch of aid when it truly is required.

The importance of measuring body temperature has been agreed by researchers. The first symptoms of many diseases start with a fever; hence an accurate body temperature is meaningful. Understanding the relationship between alertness, performance and body temperature can provide important data on treatment effectiveness. Information on body temperature has become so indispensable and an unobtrusive and accurate measurement approach would be especially valuable for the higher risk elderly population.

Hence there is a need for a method and system that is capable of overcoming one or more of the above identified challenges.

### SUMMARY OF THE INVENTION

Our invention solves the problems of lost dental appliances, dental compliance, body temperature trending, as well as identifying falls, impact, or trauma to the individuals wearing the appliances.

In particular, and by way of example only, according to at least one embodiment, provided is a smart dental unit (SDU) restoring one or more teeth, including: a prosthetic base structured and arranged to engage with mouth tissue, the prosthetic base supporting at least one tooth and at least one sensor system, the at least one sensor system including: at least one unique identifier, structured and arranged to identify the SDU; at least one sensor, structured and arranged to detect at least one event; a processor; a non-volatile memory coupled to the processor having processor executable instructions to direct the operation of the processor and the at least one sensor for detection of one or more events experienced by the SDU; a wireless transceiver coupled to the processor and the non-volatile memory and operable to communicate with at least one remote computing system; and a power source structured and arranged to power at least the processor, the at least one sensor and wireless transceiver; and wherein in response to the detection of one or more events by the at least one sensor, the processor generating data communicated by the wireless transceiver to the remote computing system.

For another embodiment, provided is a smart dental unit (SDU) restoring one or more teeth, including: a prosthetic base structured and arranged to engage with mouth tissue, the prosthetic base supporting at least one tooth and at least one sensor system, the at least one sensor system including: at least one unique identifier, structured and arranged to identify the SDU; at least one accelerometer; at least one thermometer; at least one pulse oximetry sensor; a processor; a non-volatile memory coupled to the processor having processor executable instructions to direct the operation at least the processor and the at least one accelerometer and thermometer for detecting a kinetic event and/or present temperature event; a wireless transceiver coupled to the processor and the non-volatile memory and operable to communicate with at least one remote computing system; a power source structured and arranged to power at least the processor, accelerometer, thermometer and wireless transceiver; and wherein in response to the detection of one or more events by the at least one sensor, the processor generating data communicated by the wireless transceiver to the remote computing system.

For still another embodiment, provided is a method providing a smart dental unit (SDU) restoring one or more teeth, including: providing a prosthetic base structured and arranged to engage with mouth tissue, the prosthetic base supporting at least one tooth and at least one sensor system, the at least one sensor system including: a sensor disposed within the smart tech repository, the sensor including: at least one unique identifier, structured and arranged to identify the SDU; at least one sensor, structured and arranged to detect at least one event; a processor; a non-volatile memory coupled to the processor having processor executable instructions to direct the operation of at least the processor and the at least one sensor for detection of one or more events experienced by the SDU; a wireless transceiver coupled to the processor and the non-volatile memory and operable to communicate with at least one remote computing system; a power source structured and arranged to power at least the processor, accelerometer, thermometer and wireless transceiver; and wherein in response to the detection of one or more events by the at least one sensor, the processor generating data communicated by the wireless transceiver to the remote computing system.

And for yet still another embodiment, provided is a system for dental based patient care, including: at least one smart dental unit (SDU) restoring one or more teeth, the SDU including a prosthetic base structured and arranged to engage with mouth tissue, the prosthetic base supporting at least one tooth and at least one sensor system, the at least one sensor system including: at least one unique identifier, structured and arranged to identify the SDU; at least one sensor, structured and arranged to detect at least one event; a processor; a non-volatile memory coupled to the processor having processor executable instructions to direct the operation of the processor and the at least one sensor for detection of one or more events experienced by the SDU; a wireless transceiver coupled to the processor and the non-volatile memory and operable to communicate with at least one remote computing system; and a power source structured and arranged to power at least the processor, the at least one sensor and wireless transceiver; and wherein in response to the detection of one or more events by the at least one sensor, the processor generating event data communicated by the wireless transceiver to the remote computing system; a plurality of position agents each having a pre-determined detection range and structured and arranged to wirelessly detect the presence of an SDU within the pre-determined detection range and report the detection to the at least one remote computing system; at least one database providing by the at least one remote computing system, the at least one database further comprising user data, SDU data, and event threshold data, the at least one computing system receiving event data from at least one SDU known to the system, the computer system further evaluating the event data to threshold data for the known SDU and issuing an alert in response to the event data is outside of the threshold data.

### BRIEF DESCRIPTION OF THE DRAWINGS

The following photographs show components and the method of fabrication of the smart dental unit (SDU) as a dental appliance such as a removable denture.

FIG. 1 illustrates a high-level diagram of a smart dental unit and system for dental based patient care in accordance with an embodiment of the present invention;

FIG. 2 illustrates a conceptual overview in block diagram format for data exchange within the smart dental unit in accordance with an embodiment of the present invention;

FIG. 3A is an enlarged side perspective view of a smart dental unit in accordance with at least one embodiment of the present invention;

FIG. 3B is an enlarged side perspective view of a smart dental unit in accordance with at least one embodiment of the present invention;

FIG. 3C is an enlarged front plane view of a smart dental unit in accordance with at least one embodiment of the present invention;

FIG. 3D is an enlarged side plane view of a smart dental unit in accordance with at least one embodiment of the present invention;

FIG. 3E is a top plane view of a smart dental unit as a partial dental appliance in accordance with at least one embodiment of the present invention;

FIG. 3F is an front perspective view of the smart dental appliance in FIG. 3E in accordance with at least one embodiment of the present invention;

FIG. 4A is an enlarged perspective bottom view of a smart dental unit in accordance with at least one embodiment of the present invention;

FIG. 4B is an enlarged perspective top view of the smart dental unit in FIG. 4A in accordance with at least one embodiment of the present invention;

FIG. 5A is an enlarged perspective bottom view of a smart dental unit in accordance with at least one embodiment of the present invention;

FIG. 5B is an enlarged perspective top view of the smart dental unit in FIG. 5A in accordance with at least one embodiment of the present invention;

FIGS. 6A-6D are perspective side views of an embodiment providing a smart dental unit in accordance with at least one embodiment of the present invention;

FIG. 7 is a an expanded conceptual view of a system for dental based patient care in accordance with an embodiment of the present invention;

FIG. 8 is a conceptual view of a facility incorporating a system for dental based patient care in accordance with an embodiment of the present invention;

FIG. 9 is a high-level block diagram of a computer system in accordance with at least one embodiment.

### DETAILED DESCRIPTION

Before proceeding with the detailed description, it is to be appreciated that the present teaching is by way of example only, not by limitation. The concepts herein are not limited to use or application with a specific system or method for kinetic or temperature sensing activity or any monitoring of a dental appliance. Thus, although the instrumentalities described herein are for the convenience of explanation shown and described with respect to exemplary embodiments, it will be understood and appreciated that the principles herein may be applied equally in other types of systems and methods involving kinetic sensing activity or any monitoring of a dental appliance.

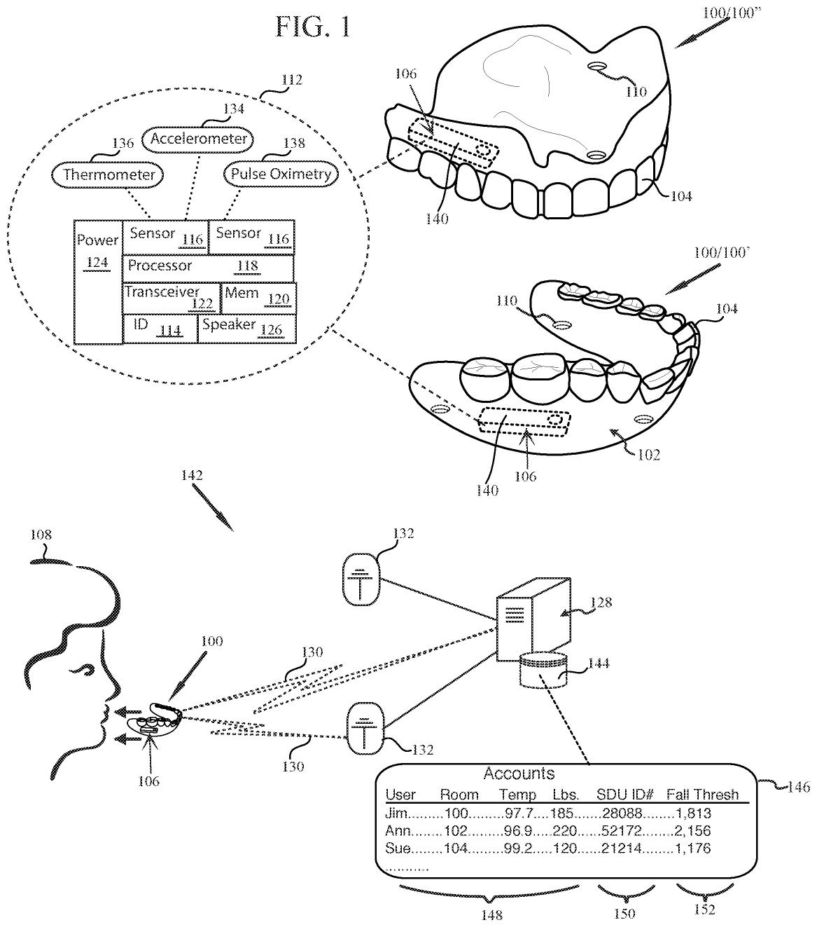

This invention is described with respect to preferred embodiments in the following description with references to the Figures, in which like numbers represent the same or similar elements. It will be appreciated that the leading values identify the Figure in which the element is first identified and described, e.g., element 100 first appears in FIG. 1.

Various embodiments presented herein are descriptive of apparatus, systems, articles of manufacturer, or the like for systems and methods for multi-modal dosing. In some embodiments, an interface, application browser, window or the like may be provided that allows the user of the computing device to direct behavior of the computing device.

Moreover, some portions of the detailed description that follows are presented in terms of the manipulation and processing of data bits within a computer memory. The steps involved with such manipulation are those requiring the manipulation of physical quantities. Generally, though not necessarily, these quantities take the form of electrical or magnetic signals capable of being stored, transferred, combined, compared and otherwise manipulated. Those skilled in the art will appreciate that these signals are commonly referred to as bits, values, element numbers or other clearly identifiable components.

It is of course understood and appreciated that all of these terms are associated with appropriate physical quantities and are merely convenient labels applied to these physical quantities. Moreover, it is appreciated that throughout the following description, the use of terms such as “processing” or “evaluating” or “receiving” or “outputting” or the like, refer to the action and processor of a computer system or similar electronic computing device that manipulates and transforms the data represented as physical (electrical) quantities within the computer system's memories into other data similarly represented as physical quantities within the computer system's memories.

The present invention also relates to an apparatus for performing the operations herein described. This apparatus may be specifically structured for the required purposes as are further described below, or the apparatus may be a general-purpose computer selectively adapted or reconfigured by one or more computer programs stored in the computer upon a computer readable storage medium suitable for storing electronic instructions.

Turning now to the figures, and more specifically FIG. 1, as will be further apparent from the following description and accompanying figures, provided is a system and method that permits monitoring of dental treatment compliance, body and appliance temperature, trauma to the head and neck, and the location of a dental appliance. Key for this advantageous system and method is a smart dental unit 100, hereinafter SDU 100, in accordance with at least one embodiment of the present invention.

As shown, SDU 100 is provided as a prosthetic base 102 supporting at least one tooth 104 and at least one sensor system 106. Moreover, in the following discussion the SDU 100 is understood and appreciated to be the entire dental appliance incorporating in varying embodiments smart technology at least one or more kinetic, temperature, and location systems, e.g., the SDU sensor system 106.

The prosthetic base 102 is structured and arranged to engage with mouth tissue (not shown) of a user 108. Moreover, the prosthetic base 102 may be formed as a dental appliance such as a denture—a removable prosthetic device for replacement of missing teeth and surrounding tissue. It will be understood and appreciated that dentures are commonly of two types—complete and partial. With a complete denture all of the patient's teeth are missing, whereas with a partial denture, some natural teeth remain.

As the human mouth has an upper set of teeth and a lower set of teeth, it will be understood and appreciated that embodiments of SDU 100 may be provided as a complete or partial denture for either the upper or lower teeth, or both, without departure from the scope and teachings herein, even though physical differences between an upper and lower dental appliance may exist. Varying embodiments of the SDU 100 of the present invention are equally advantageous and adaptable for either complete or partial dentures.

It will also be understood and appreciated that to some users, a partial denture may be referred to as a “bridge” which those in the dental arts will typically understand to be permanent prosthetic device. Although an embodiment, of SDU 100 may be intended as a permanent or semi-permanent dental prosthesis, for advantageous simplicity of recharging and/or for improved patient hygiene, it will be understood and appreciated that other embodiments of SDU 100 are removable such that the internal sensor system 106 may be recharged and the SDU 100 cleaned on a daily basis—typically overnight—as is typical with prosthetic dentures.

Removable embodiments of SDU 100 may optionally incorporate at least one mount 110 structured and arranged to engage with at least one anchor (not shown) disposed within a person's mouth. For example, embedded snap rings may be disposed in the prosthetic base 102 so as to align with anchor pins that have been surgically disposed within a user's mouth. The use of at least one mount 110 may serve to not only secure the SDU 100 for normal use, but may advantageously ensure that elements of the sensor system 106 are optimally disposed within the person's mouth for the measurement of biometric factors.

As shown, FIG. 1 presents an embodiment of SDU 100 as a lower denture SDU 100′, and an upper denture SDU 100″. From these illustrations it may be further appreciated that the lower denture SDU 100′ is essentially in the shape of a “U” with an open middle section so as to accommodate the user's tongue, while the upper denture SDU 100″ has a solid middle section which is typically intended to be disposed against the user's upper pallet.

As the rear of the upper or lower denture is typically wider—corresponding with the typical morphology of a person's mouth, the sensor system 106 is typically intended for installation within the prosthetic base 102 under one or more rear molar teeth. Moreover, the SDU 100 will be understood and appreciated as a dental appliance that fits entirely within the person's mouth. For the embodiments shown and described herein, the embodiments discussed are generally with respect to full dentures for ease of illustration. However, it will be further understood and appreciated that varying embodiments of an SDU 100 may incorporate essentially any dental appliance that fits within a person's mouth, e.g., mouthguards such as may be used to Bruxism or TMJ disorder as is often caused by teeth grinding. Moreover, although the teachings of the present invention are applicable to dental prosthesis appliances, the teachings are also applicable to corrective dental appliances as well as protective dental appliances.

With respect to FIG. 1, dotted bubble 112 presents a conceptual illustration of the elements comprising the sensor system 106. More specifically, for at least one embedment, the sensor system 106 comprises at least one unique identifier 114, structured and arranged to identify the SDU 100; at least one sensor 116, structured and arranged to detect at least one event; a processor 118; non-volatile memory 120 coupled to the processor 118; a wireless transceiver 122; and a power source 124. For at least one optional embodiment the sensor system 106 may also include an audible alert element, such as a speaker 126.

As at least the processor 118, sensor 116, and wireless transceiver 122 are understood and appreciated to be electrical devices, the SDU 100 and more specifically the sensor system 106 are further understood and appreciated to include a power source 124. For at least one embodiment the power source 124 may be an integrated component of the sensor system 106, and more specifically an integrated component of the SDU chip 140. For yet other embodiments, the power source 124 is a separate embedded element eclectically coupled to the sensor system 106, such as by electrical lines disposed within the SDU 100.

For at least one embodiment the power source 124 is rechargeable, by induction from an external source, or by direct connection, such as through a port, such as but not limited to a USB port, provided with a sealing closure in the exterior of the prosthetic base 102. As recharging by induction permits greater moisture resistance by permitting the SDU 100 to be provided as an entirely sealed device, which also does not require the operator to have dexterity sufficient to open/connect/reseal small components, it is anticipated that embodiments of SDU 100 with inductively recharged power source 124 will likely be preferred.

The non-volatile memory 120 provides executable instructions to direct the operation of the processor 118 and the at least one sensor 116 for the detection of one or more events experienced by the SDU 100. The wireless transceiver 122 is also coupled to the processor and the non-volatile memory and operable to communicate with at least one remote computing system.

For at least one embodiment the wireless transceiver 122 operates in the form of Bluetooth Low Energy (BLE). For at least one alternative embodiment the wireless transceiver 122 operates in the form of near field communication (NFC). For yet another embodiment the wireless transceiver 122 operates in the form of cellular communication (LTE, 4G, 3G). For at least one embodiment, the wireless transceiver 122 is a wireless network component.

Moreover, the wireless transceiver 122 is understood and appreciated to permit the wireless transfer of data as between the SDU 100 and the at least one remote computing system 128. Further still, for at least one embodiment the wireless transceiver 122 is structured and arranged to use existing wireless data technologies such that one or more SDUs 100 may be quickly and advantageously deployed in a new or existing environment without substantial burden to the users and caregivers.

As is further discussed below, although each SDU 100 may be capable of wireless communication 130 directly with the at least one remote computing system 128, for at least one embodiment, such communication may be indirect in that an SDU 100 may engage in wireless communication 130 with a wireless access point 132 which in turn is in data communication with the at least one remote computing system 128. By determining which wireless access point 132, or remote computing system 128 an SDU 100 is presently using (or last used) an approximation of the SDU 100 may be determined. Further still, the use of specific near field wireless access points may advantageously permit improved precision in determining the location of any given SDU 100.

In varying embodiments, the at least one sensor 116 is selected from the group consisting of, but not limited to an accelerometer 134, a thermometer 136, and a pulse oximetry sensor 138. It will be understood and appreciated that in varying embodiments, additional types of sensors 116 may be incorporated beyond those noted above, when and as such additional sensors are deemed beneficial. With respect to the term “sensor” as used herein, it is to be understood and appreciated that a sensor is a device, module, component or subsystem having the purpose to detect events or changes in the environment and provide such information to other electronics within the SDU 100 system. As the sensor is “detecting” events, it shall be understood and appreciated that as used herein, or with respect to the identification of an accelerometer 134, a thermometer 136, a pulse oximetry sensor 138, the term sensor or detector may be used without departure from the teachings herein unless expressly so stated.

As is further described herein, embodiments of the SDU 100 advantageously permit improved trauma monitoring and assessment, such as for the detection of trauma from a blow or an unsupervised individual experiencing a fall, as well as dental treatment compliance for the user 108 of the SDU 100. This is achieved at least in part through the use of at least one remote computing system 128, such as a desktop, smartphone, or other computing device which may be adapted by hardware or software application(s) for wireless communication 130 with the SDU 100 to track the location of one or more SDU 100 devices, and receive information from the sensor system 106 of each SDU 100, and thus at least some measure of the current condition of any given user 108 of an SDU 100 known to the remote computing system 128.

Although such falls are perhaps most common with the elderly, various medical conditions are known which can befall people of all ages, such that it is understood and appreciated that the benefits of an SDU 100 in detecting a fall that is not otherwise observed by another human, is highly advantageous in the pursuit of high-quality medical care and patient wellbeing.

The advantages of SDU 100 are not limited to the detection of physical trauma. Dental treatment compliance is important for the elderly in ensuring the dental appliance is used according to the prescribed dental treatment, normally daily use with nightly removal and cleaning. Non-compliance can result in poor oral hygiene which can impact a person's overall health. With the significant difference in oral versus room/external, temperature trending as permitted by embodiments of SDU 100 configured with a thermometer component of the sensor system 106 provides key data on use of the dental appliance.

For at least some embodiments, the sensor system 106 may be provided as a single chip or printed circuit board (PCB) that has been formed substantially as a unitary sensor system 106, i.e., the “SDU chip 140.” For at least some embodiments, the use of an SDU chip 140 may be advantageous in permitting easy and quick modification of an existing prosthetic base 102.

For yet other embodiments, enhanced sensing capabilities, range of wireless transmission, extended operating time between recharging, the size of the patient's mouth, or other factors may be improved beyond those of a single SDU chip 140 by distributing some or all of the sensor system 106 components within the prosthetic base 102. Moreover, for at least one embodiment, the sensor system 106 may be established by electrically interconnecting components embedded within the prosthetic base 102, such as a distinct battery, an antenna and processor with sensor.

Whether provided as a SDU chip 140 or potentially distributed sensor system 106, various embodiments of SDU 100 provide kinetic sensing (can detect fall or sudden impact), have temperature sensing (can detect dental appliance use and potential health issues), and permit tracking (can be found remotely), as well as provide the advantageous ability to detect pulse oximetry (oxygen in the blood stream as well as pulse rate). It should be understood and appreciated that in various embodiments, some of these detected/determined data points may be collectively provided, or subsets of same preferred.

Moreover, the SDU 100 provides a highly advantageous platform for the improvement of patient care by detecting and reporting events in near real time that pertain to the patient's general health, beyond the traditional functions of a dental appliance to permit improved chewing and eating as well as perhaps elevated self-esteem from a pleasant smile.

Returning to FIG. 1, and general embodiments for SDU 100, the unique identifier 114 is to uniquely identify the SDU 100. Such a unique identifier 114 may be achieved by RFID, BLE, EEPROM or other element that may be stand alone or be incorporated as a component of another element noted above. This unique identifier is typically established for the SDU 100 during manufacturing of the SDU 100, or at the sensor system 106 that may be implanted into a prosthetic base 102.

The manufacturing process will assign, and then permanently set this unique identifier on the SDU 100 hardware. The unique identifier 114 permits the SDU 100 belonging to patient “Jim” to be distinguished from the SDU 100 belonging to patient “Sue”, the SDU 100 belonging to patient “Ann” etc . . . . For at least one embodiment, each unique identifier 114 is indeed unique such that each and every SDU 100 can be distinguished from every other SDU 100.

In other embodiments, each SDU 100 is uniquely identifiable by programming as performed by an enabled operator—but programming may permit multiple SDUs to be programmed with the same apparent unique identifier 114. For example, a SDU 100 in Denver, Colo. might be identified as “Bob's appliance” and a similar SDU 100 in Orlando Fla. might belong to a different Bob, but be identified as “Bob's appliance.” As each SDU 100 device would be operating in a different environment, such confusion would not be a significant issue for the system.

For yet another embodiment, each SDU 100 may be uniquely identified, but permit an enabled user to provide a custom tag as well—such as the owner's name, i.e., “Jim-28088.” It should also be understood and appreciated that the RFID, BLE, EEPROM or other element may be referred to as a tag, chip or component. Moreover, the unique identifier may be a distinct element, or an element that is integrated as a part of another component.

With respect to FIG. 1, it will be further understood and appreciated that the SDU 100 and the at least the one remote computing system 128 form the core of an advantageous System For Dental Based Patient Care 142, hereinafter SDBP 142. For at least one embodiment of SDBP 142, such as where a plurality of users 108 have associated SDUs 100, the remote computing system 128 has a database 144, and hardware or software adapting the at least one remote computing system 128 to receive data regarding detected events from each SDU 100 known to the system. Portable computing devices, such as an iPhone® or an Android® (not shown) may also be adapted by software application to participate in SDBP 142 as, but not limited to, portable alert devices for caregivers, input/output devices or SDBP 142 configuration and adjustment, portable detectors for SDUs 100 within an environment governed by an SDBP 142, or such other configurations as are deemed advantageously appropriate.

The database 144 may be configured in a variety of different ways, and for ease of illustration and discussion has been conceptually shown to provide records 146 of the assigned user 108 (user data 2) (e.g. name, room, normal temperature, weight, etc. . . . ), SDU data 150 for each SDU 100 as related to a known user 108, (e.g., the unique identifier of the associated SDU 100), and such other data as may be desired—such as but not limited to the user's times of eating and sleeping, the user's typical temperature/blood pressure/heart rate/etc . . . . , and thresholds for events 152.

For the example shown, an exemplary threshold of force representing a kinetic event of a fall has been shown based on the basic formula of Force=Mass*Acceleration, wherein Mass=exemplary body weight and Acceleration equal Gravity (e.g., 9.8 m/s\hat{ }2). This exemplary value is used for discussion proposes only, and it is understood and appreciated that varying equations and proprietary methodologies may be advantageously incorporated for very precise determination of, and distinction between various kinematic events, such as falls/sitting/chewing/talking/etc . . . .

It will also be appreciated that the database 144 may also be an entirely local database or a distributed database, and may also be a flat file, relational database, or such other form as is deemed most appropriate for a particular embedment of SDBP 142. In addition, the exemplary distinctions between user data 148 and SDU data 150 as presented herein are for ease of illustration and discussion, and not intended as limitations. Moreover, the data present in the database 144 regarding users 108, SDU 100 and any other factors or elements pertaining to a specific or general environment wherein SDBP 142 is implemented may be adapted and/or configured as deemed appropriate for each specific instance of SDBP 142.

It will also be understood and appreciated that the remote computing system 128 may in varying embodiments be a plurality of different computing systems, and/or incorporate additional remote or cloud-based computing resources. In addition, the database may be a local database or distributed database, and information as collected from the use of one or more SDU 100 devices may be shared with third parties for a variety of purposes including, but not limited to verification of patient care and refinements for improvement of patient care.

As the unique identifier 114 is described as being provided by RFID, BLE, EEPROM or other transmissive/responsive element, it will be understood and appreciated that the determination of an SDU 100 unique identifier 114 will typically be determined electronically. While each SDU 100 may indeed have a visually determinable unique identifier (e.g., a printed serial number) as used herein the unique identifier 114 is understood and appreciated to be determined remotely by electronic and/or remote computing systems in the environment wherein the user 108 of the SDU 100 lives, works, or otherwise spends time. This remote determination of each SDU 100, and thereby the user 108 as well, advantageously aids in the ability to monitor a user's location as well as status in substantially real time.

Moreover, with respect to the above description, at least one embodiment of the present invention for a SDU 100 may be summarized as a prosthetic base 102 structured and arranged to engage with mouth tissue, the prosthetic base 102 supporting at least one tooth 104 and at least one sensor system 106, the at least one sensor system 106 including: at least one unique identifier 114, structured and arranged to identify the SDU 100; at least one sensor 116, structured and arranged to detect at least one event; a processor 118; a non-volatile memory 120 coupled to the processor 118 having processor 118 executable instructions to direct the operation of the processor 118 and the at least one sensor 116 for detection of one or more events experienced by the SDU 100; a wireless transceiver 122 coupled to the processor 118 and the non-volatile memory 120 and operable to communicate with at least one remote computing system 128; and a power source 124 structured and arranged to power at least the processor 118, the at least one sensor 116 and wireless transceiver 122. In response to the detection of one or more events by the at least one sensor 116, the processor 118 generating data communicated by the wireless transceiver 122 to the remote computing system 128.

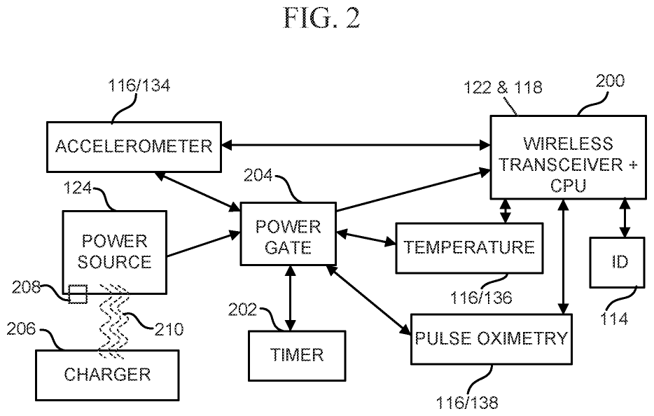

FIG. 2 presents a conceptual overview in block diagram format for the interactions and exchanges of data for at least one embodiment of SDU 100, and more specifically at least one embodiment of the sensor system 106. Continuing from the overview above, for at least one embodiment the sensor system 106 includes a processor 118, non-volatile memory 120 and wireless transceiver 122, which in FIG. 2 have been shown as a collective CPU element 200.

For the embodiment shown in FIG. 2, the sensor system 106 includes three sensors 116; an accelerometer 134, a thermometer 136, and a pulse oximetry sensor 138. A timer 202 and power gate 204 are also provided, and an external charger 206 is shown which may recharge the power source 124 by direct connection to an optional port 208, or wireless induction coupling shown by wavy lines 210.

Of course, it is understood and appreciated that each of these elements may in turn be comprised of multiple sub-elements, and/or may share sub-elements, and in some cases may be embodied by physical systems structured for a specific purpose, or may be more generalized components that are adapted by software or firmware to achieve a specific purpose as one of the identified elements, or component thereof.

For at least one embodiment the thermometer 136 is a MAX30208CLB+ temperature sensor as provided by Maximum Integrated (https://www.maximintegrated.com/en.html), the pulse oximetry sensor 138 is, or is adapted from, a MAXREFDES117# Eval BRD Heart Oximetry Monitor from Maximum Integrated, the at least one embodiment the accelerometer 134 is a LSM5DS032TR memo Internal Module: 3D Accele (motion sensor) from STMicroelectrics (https://www.st.com/content/st_com/en.html), and the wireless transceiver 200 is an NRF52811-CAAA-R IC Bluetooth transceiver from Nordic Semiconductor ASC (https://www.nordicsemi.com)—each of these elements available from DigiKey Electronics (https://www.digikey.com).

In varying embodiments, some or all of these components are incorporated into the SDU chip 140 as discussed above, and which is incorporated into or below prosthetic teeth to be inserted during the fabrication of the SDU 100 prosthetic appliance or in the retrofitting of a current prosthetic appliance to become an SDU 100. Further still, it will be appreciated that although a single SDU chip 140 may be a common embodiment, for yet other embodiments it may be desirable, if not advantageous to have the elements herein noted divided between one or more PCB's, chips, or other structural elements distributed throughout the removable prosthetic base 102 of the SDU 100.

For at least one embodiment, such as to conserve power and/or extend operation between charging sessions or battery replacement, systems within the SDU 100, and more specifically the sensor system 106, may be operated in various modes from all to very few. In other words, the processor 118 may shunt power to one or more system or sensors 116 that is/are not currently in use or required for a current operation. For example, the processor 118 may maintain operation and data exchange with the accelerometer 134 to detect a kinetic event, but effectively disconnect the wireless transceiver 122 until there is data to transmit, or it is time for the SDU 100 to check in, or another event. This same control may be extended to the temperature sensor (thermometer 136), and other systems that may be provided in various embodiments, but not required in an ever-present “on/powered up” state unless directed so by a user.

For yet another embodiment, as is shown in FIG. 2, power for the SDU 100 is provided by the internal power source 124 (i.e., a battery) to the power gate 204 which governs allocation of power to the processor 118 and wireless transceiver 122 (CPU element 200). The purpose of the power gate 204 is to only allow power to be delivered to the rest of the sensor system 106 when it is needed. For at least one embodiment, determination of when power is needed is done with the power gate algorithm. One simple method of accomplishing this is to use a timer 202 to periodically open the power gate 204 and allow power to be delivered to the rest of the sensor system 106. For yet another embodiment, the power gate 204 may be triggered by accelerometer detections. Moreover, although for at least one embedment the power gate 204 may be omitted, for at least one embodiment, improved longevity of performance is permitted by the incorporation of a power gate 204 to effectively govern and manage the usage of power by the elements of the sensor system 106 of SDU 100.

The unique identifier 114 may be understood and appreciated to identify a given SDU 100 in a variety of different ways. In addition to being potentially provided in one or more embodiments as a passive or active RFID chip or tag, the unique identifier may also be encoded by the processor 118/CPU element 200 in all data transmissions, such as in a header tag, packet, file or other data structure. Such data tagging may be advantageous in easily establishing an archive of data and/or events specific for each known SDU 100.

Those skilled in the art will understand and appreciate that passive RFID tags/chips passively react to near field electromagnetic waves and therefore require no internal power source. For environments where an SDU 100 user may pass through doorways or other narrow spaces frequently, such passive RFID implementation may be advantageous in permitting at least some instances of SDU 100 identification without power drain from the power source 124. However, an active RFID chip or tag may be more advantageous for identifying the locations of one or more SDUs 100 in more open settings, and may in some embodiments be configured for active response without necessitating full activation of all components within the sensor system 106.

It will be understood and appreciated that varying embodiments of SDU 100 may provide data to the remote computing system 128 under a variety of circumstances. For embodiments of SDU 100 incorporating the unique identifier as an RFID chip or tag, the proximity of the RFID chip or tag to a sensor within the environment may trigger the remote computer to poll/trigger the SDU 100 to send data. The SDU 100 may also be structured and arranged to transmit data at regular time intervals—i.e., every 10 seconds/1 minute/14 minutes/30 minutes/etc. . . . or other time intervals as deemed appropriate. The SDU 100 may also be structured and arranged to transmit data when a detected event is above a pre-determined threshold, such as a kinematic event wherein the acceleration or deceleration is outside of a pre-established range, a temperature reading that is above or below a pre-established range, a humidity level that is above or below a pre-established range, a pulse oximetry value that is above or below a pre-established range, etc . . . .

Although the SDU 100 may be structed and arranged to detect an event—such as a kinematic event and initiate a transfer of data to the remote computer regarding such detection, for at least one embodiment, the SDU 100 may operate primarily as a data collector and reporter. Moreover, for ease of fabrication, reduction of costs, and potentially improved longevity of the sensor system 106, the remote computing system 128 may be adapted by software and or hardware to processes data provided by the SDU 100 for the actual identification and detection of specific events generalized as baselines for a plurality of users, or specifically tailored to each user 108. This may include, but is not limited to, the user's exercise patterns, eating pattern, sleeping pattern, hydration patterns, and the like.

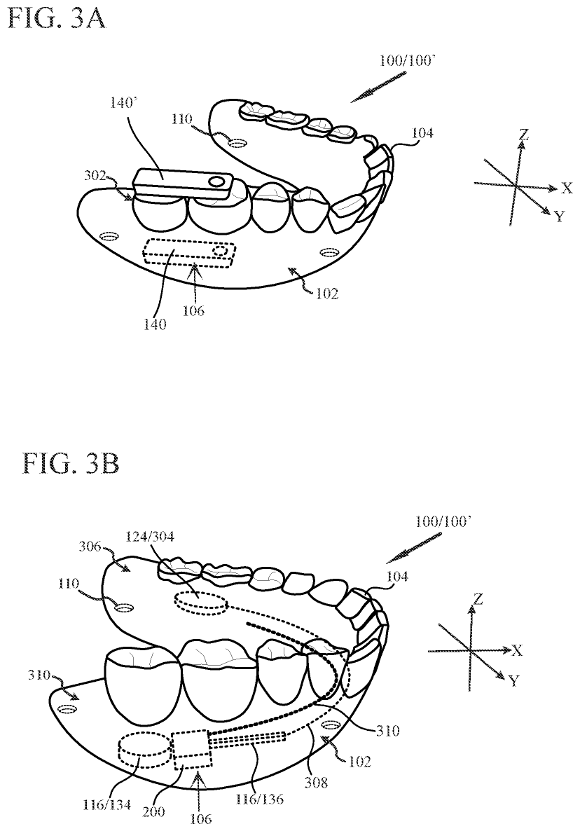

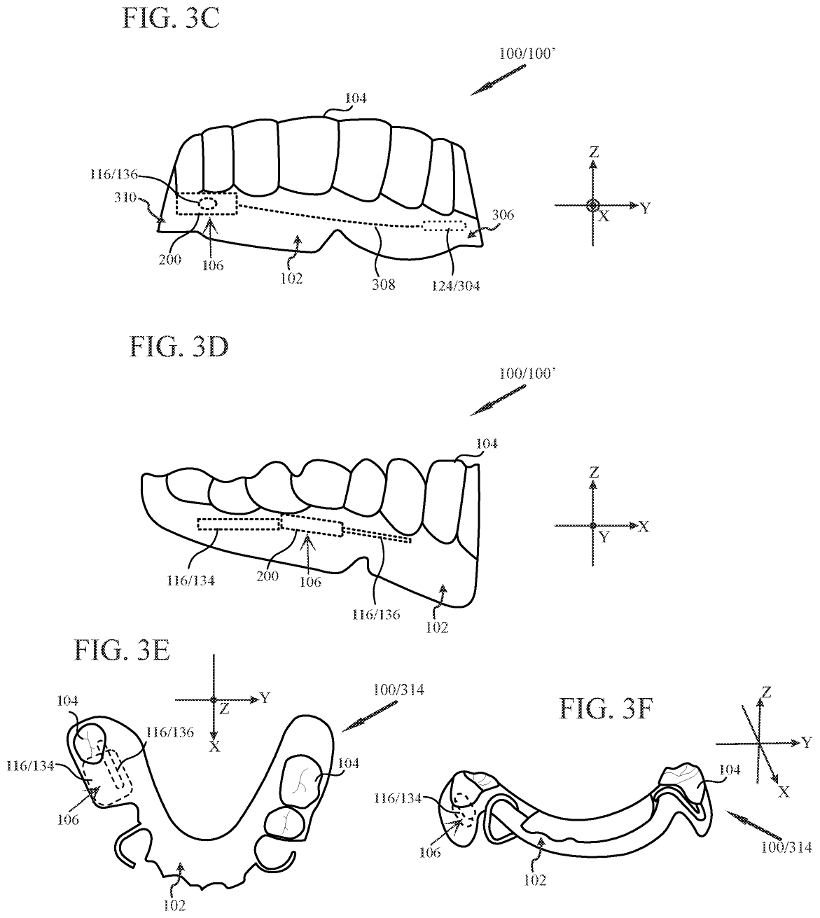

FIGS. 3A through 5B present additional views of varying embodiments for SDU 100 with respect to the upper denture, lower denture, and configurations of sensor system 106. To facilitate the description of systems and methods for embodiments of SDU 100, the orientation of SDU 100 as presented in the figures is referenced to the coordinate system with three axes orthogonal to one another as shown in FIG. 3A. The axes intersect mutually at the origin of the coordinate system, which is chosen to be the center of SDU 100, however the axes shown in all figures are offset from their actual locations for clarity and ease of illustration.

FIG. 3A provides an enlarged perspective view of an SDU 100 as a bottom denture 300, with an SDU chip 140′ disposed upon the two back right teeth 302 for a conceptualization of scale for at least one embodiment of the sensor system 106. In FIG. 3A an SDU chip 140 is shown in dotted relief as embedded within the prosthetic base 102. For at least one embodiment, the SDU chip 140 has the physical dimensions of about 4 mm×4 mm×16 mm.

FIG. 3B is a similar perspective view of an SDU 100 as a bottom denture with the sensor system 106 as a distributed system within the prosthetic base 102. More specifically, the battery 304 is shown disposed within the left side 306 of the prosthetic base 102 with at least one electrical wire 308 interconnecting the battery 304 to the CPU element 200 and associated sensors 116 (an accelerometer 134 136), shown disposed in the right side 310 of prosthetic base 102.

For at least one embodiment, the antenna may be integrated with the electrical wire 308, or as shown it may exist as a separate antenna 312. FIG. 3C presents a front view of the SDU 100 shown in FIG. 3B and FIG. 3D presents a side plane view of the SDU 100 shown in FIG. 3B. In further demonstration of embodiments of SDU 100 for other than full dentures, FIGS. 3E and 3F present a top and front perspective view of a partial dental appliance, depicted as partial bottom appliance 314. For all of these and the following figures, it is to be understood and appreciated that the order of illustration and identification for the components has been chosen for ease of illustration and discussion, not an intended limitation.

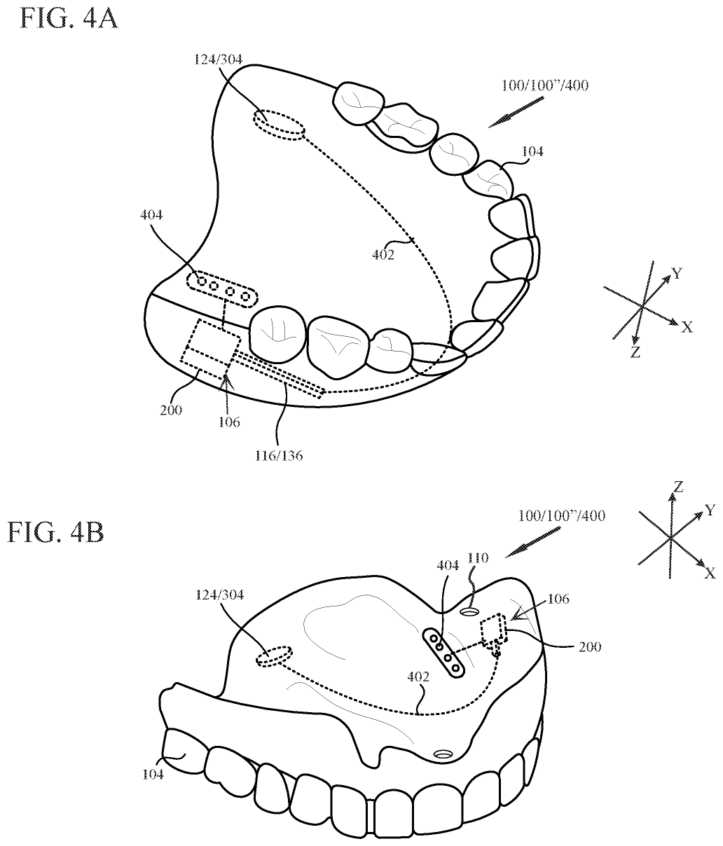

FIG. 4A presents a perspective bottom view of an embodiment of SDU 100/100″ as an upper denture 400, and FIG. 4B presents a top perspective view. For the embodiment as illustrated, the sensor system 106 is distributed with the power source 124 (battery 304) disposed on one side, and the CPU element 200 and sensor 116 disposed on the other with at least one electrical line 402 therebetween.

For this embodiment as shown, a plurality of contact sensors 404 are also shown. These contact sensors 404 are understood and appreciated to be disposed in the outer surface of the prosthetic base 102 in areas that will be in physical contact with the user's mouth tissue. More specifically, these contact sensors 404 are intended to press against the mouth tissue of the user when the SDU 100 has been placed in the mouth. In varying embodiments these contact sensors 404, can detect moisture and temperature. For yet other embodiments the contact sensors may also detect salinity, Ph level, and/or such other characteristics of fluids within the mouth as may be deemed desirable.

Although temperature may be determined by one or more sensors disposed more deeply within the prosthetic base 102, for at least one embodiment the use of contact sensors 404 in place of, or in addition to, deeper sensors may be desired for faster and/or more precise sensing. For at least one embodiment moisture sensing may be desired to further monitor and maintain proper hydration of the user.

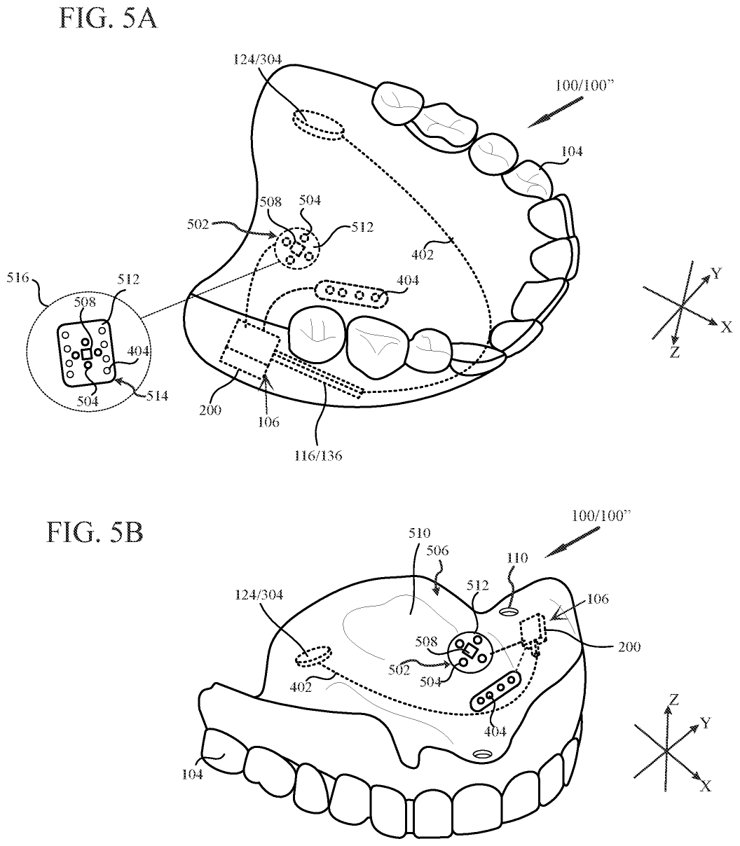

FIGS. 5A and 5B present a similar perspective bottom view and top plane view of yet another embodiment of SDU 100/100″/500 as an upper denture incorporating a pulse oximetry sensor 502. It is to be understood and appreciated that pulse oximetry is a non-invasive method used to monitor the oxygenation level of the blood, a non-invasive procedure typically involving light. For such an embodiment, typically one or more LEDs 504 are provided within or disposed at least partially upon or in close proximity to the outer surface 506 of the SDU 100 that is proximate to mouth tissue. For at least one embodiment, the LEDs 504 are disposed about at least one optic sensor 508.

For the illustrated example, the pulse oximetry sensor 502 is disposed in the central portion 510 of the top of the prosthetic base 102. The areas adjacent to the LEDs 504 and optic sensor 508 may be coated with a light-blocking, light filtering or light absorbing coating so as to aid in allowing only certain wavelengths of light (those emitted from one or more of the LEDs) to reach the optic sensor 508. For at least one embodiment, the LEDs 504 of the SDU 100 emit light at 424 or 624 nanometers (nm), but other wavelengths of light known to those skilled in the art as suitable for Photoplethysmography (PPG), or pulse oximetry are within the scope of the present invention. PPG is an optical technique that measures blood volume variations, non-invasively and in substantially real time.

For at least one embedment, one or more LEDs 504 may be positioned so as to direct light through tissue of the patients mouth and into the optic sensor for transmissive pulse oximetry. For at least one alternative embodiment, the LEDs and optic sensor are structured and arranged as a reflectance pulse oximetry system. Reflectance pulse oximetry does not require a thin section of the persons tissue (containing blood vessels therein) to be disposed between the LED(s) and optic sensor, and therefore may be simpler for implantation in embodiments of the present invention.

For embodiments incorporating reflectance PPG or pulse oximetry, the LEDs 504 are capable of emitting a plurality of wavelengths known in the art to be suitable for reflective PPG or pulse oximetry, such as but not limited to 424, 624, 650 and 940 nm. It will be further understood and appreciated that the dual PPG and pulse oximetry functions permit the SDU 100 with the advantageous ability to calculate any or all of the cardiovascular health markers of, but not limited to, heart rate, blood pressure, arterial stiffness, pulse transit time, pulse wave velocity, and of course blood oxygen level.

For at least one embodiment four (4) LEDs 504 are configured to symmetrically and equidistantly surround the optic sensor 508 so as to provide and maintain even light distribution for reflectance and detection. Of course, it will be understood and appreciated that similar light emitter configurations around an optic sensor may be employed and perhaps even desired for specific embodiments of SDU 100 and are therefore understood and contemplated by the present invention, including less than 4 LEDs 504. To continue with the exemplary embodiment of four LEDs, for such an embodiment a spacing of about 2 mm from the optic sensor 508 and are suitably angled—physically or by optical lens, guide, incidence angle or the like—to direct light most ideally into a person's tissue for reflection to the optic sensor. Moreover, various spacing and angles are understood to be possible and within the teaching of this specification.

With respect to the issue of pulse oximetry, it will be understood and appreciated that hemoglobin (Hb) absorbs light, and the amount of light absorbed is proportional to the concentration of Hb in the blood vessel. Beer's Law, also known as Beer-Lambert Law or the Ber-Lambert-Bouguer Law relates the attenuation of light to the properties of the material through which the light is passing. More simply stated, it is generally understood that the amount of light absorbed is proportional to the concentration of the light absorbing substance present. The light absorbed is also proportional to the length of the path the light travels.

It has also been determined that oxyhemoglobin absorbs more infrared light than red light and that deoxyhemoglobin absorbs more red light than infrared light, and red light has a different wavelength from infrared light. The absorption of green light varies as well. The use of multiple light wavelengths (red light, infrared light, and green light) and comparisons between the levels detected by one or more optic sensors may advantageously improve the accuracy of determining pulse oximetry.

For ease of assembly and alignment of the LEDs 504 and optic sensor 508, for at least one embodiment, the pulse oximetry sensor 502 is essentially pre-fabricated on an embeddable substrate 512. For at least one embodiment, the embeddable substrate 512 is a flexible element such that it may be adapted to match the topography of the user's mouth in the area where the pulse oximetry sensor 502 will be disposed by the SDU 100. For yet other embodiments, the embeddable substrate 512 may be custom formed.

As the LEDs 504 and optic sensor 508 of the pulse oximetry sensor 502 are intentionally disposed at or very close to the outer surface 506 of the prosthetic base 102, for at least one embodiment the pulse oximetry sensor 502 may also include one or more contact sensors 404 as described above, as such contact sensors 404 may be pre-fabricated on the same embeddable substrate 512 (see optional pulse oximetry sensor 502 with contact sensors 404 chip 514 in optional callout 516), or the contact sensors 404 may be disposed separately as is shown in FIGS. 5A and 5B.

With respect to the above discussions and illustrations regarding FIGS. 3A-5B, it will be understood and appreciated that the elements of the sensor system 106 are in general disposed under or otherwise displaced away from direct contact with any of the teeth 104 provided by the SDU 100. As the teeth 104 are generally made of a harder material than is the prosthetic base 102, to accommodate chewing and biting, for most embodiments it is clearly advantageous not to physically alter the teeth 104, or to dispose the sensor system 106 in such a location where possible interaction between the teeth 104 might inadvertently impinge upon the embedded sensor system 106.

It will be understood and appreciated that the mouth of any given user 108 is unique. As such, it is to be understood and expected that varying embodiments of an SDU 100 may be adaptively provided on a case-by-case basis for a user 108. For example, a user's mouth can be scanned such as by a traditional dental imaging system to provide a model for 3D printing or casting of the desired denture appliance. As such a denture appliance is cast or printed, the elements of the SDU sensor system 106 may be incorporated directly during the fabrication process.

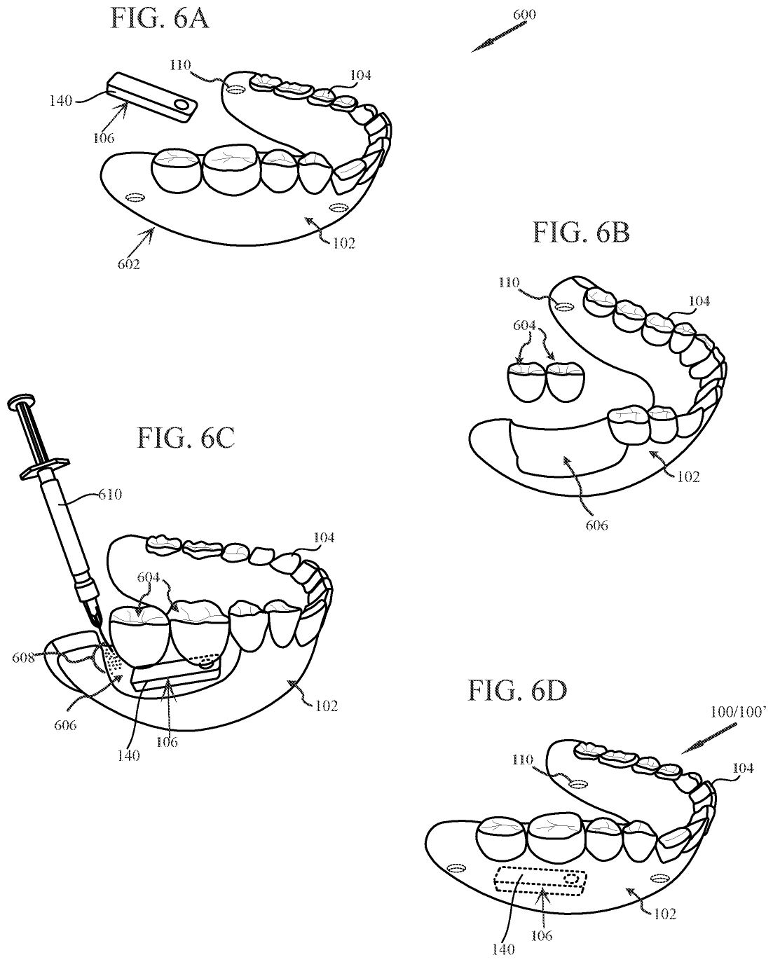

For yet another embodiment, an existing dental appliance may be modified by the temporary removal of one or more teeth or other sections to create spaces—e.g., one or more “technology repositories” for the SDU sensor system 106. FIGS. 6A-6D illustrate an exemplary process for the fabrication of an SDU 100, and for the example as shown, the sensor system 106 is provided as an SDU chip 140.

For at least one embodiment, the method 600 of providing a SDU 100, commences with an existing dental appliance such as bottom denture 602 as shown in FIG. 6A. Bottom denture 602 provides a prosthetic base 102 and at least one tooth 104. The sensor system 106, shown as an SDU chip 140 for simplicity is also provided.

Method 600 continues as shown in FIG. 6B with the removal of one or more teeth 604 and the creation of a “tech repository 606”, understood and appreciated as a cavity within the prosthetic base 102 of sufficient size and configuration to receive the sensor system 106. It will be further understood and appreciated that the choice of which teeth, and indeed the number of teeth to be removed will generally be determined on a case by case basic with respect to the size and configuration of the dental appliance being modified and the size and nature of the sensor system 106 to be embedded. As noted above, generally one or more rear teeth will be preferred as for most people this area of a dental appliance is larger and provides more space.

As shown in FIG. 6C, method 600 progresses with the sensor system 106, e.g., SDU chip 140, being disposed within the tech repository 606 and the removed teeth 604 are replaced as well. An appropriate dental acrylic or other biocompatible resin 608 is then disposed from a reservoir 610 (shown as a syringe) to fill the tech repository 606 and substantially restore the exterior appearance and surface features of the prosthetic base 102. Moreover, for the embodiment as shown, the sensor system 106 is sealed within the prosthetic base 102. As shown in FIG. 6D, method 600 results in SDU 100/100′ which is substantially as discussed and described above.

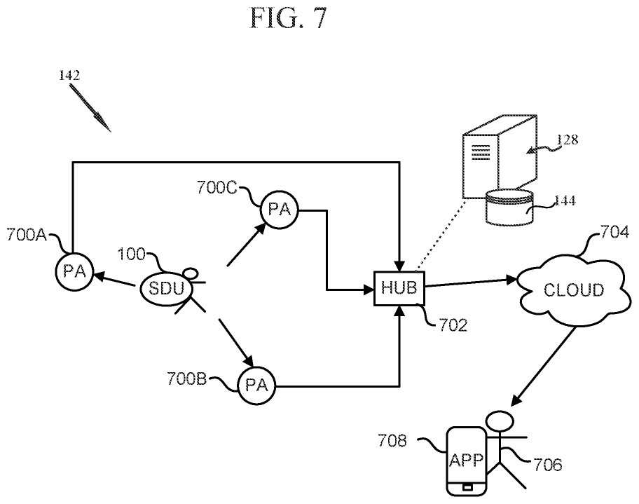

FIG. 7 presents a conceptual illustration to further assist in appreciating use of an embodiment of SDU 100. As has been set forth above, the SDU 100 is essentially a smart device capable of detecting and communicating to a remote computing system 128 at least one event. As has also been noted, the SDU 100 is advantageously beneficial for elevating patient care, ensuring compliance with prescribed dental appliance treatment, and reducing loss of dental prosthesis.

As indicated with respect to FIG. 1 as discussed above, for at least one embodiment of SDBP 142, the location of SDU 100 is facilitated by the use of access points 132, which for purposes of SDBP 142 may be further considered as Position Agents 700 (hereinafter PA(s) 700). It will be understood and appreciated that each PA 700 uses the transmitted signal from the SDU 100, or at the very least the response signal from an RFID chip or tag, to assist with determining the location of the SDU 100.

As each PA 700 may be structured and arranged as a near field device, it will be understood and appreciated that for a given PA 700 to be detecting a SDU 100 the SDU must be in close proximity to the PA 700. Moreover, it is to be understood and appreciated that for at least one embodiment, each PA 700 has a communication range that is significantly less than a typical home or business WiFi system, which may have an operating range spanning many rooms, if not hundreds of feet. Although multiple receivers can indeed be used for traditional triangulation of any given SDU 100, for at least one embodiment, it will be understood and appreciated that as the SDU 100 is moved about in an environment the SDU 100 will lose connection with one PA 700 as it moves into range with yet another PA 700.

In other words, a facility may be subdivided into different physical areas, each monitored by a PA 700—PA 700A in area 1, PA 700B in area 2, PA 700C in area 3, etc . . . . It will be understood and appreciated that each area may indeed be a bounded area, such as a bedroom, or a region within a larger space, such as subdivided areas within a large dining room or theater.

Each PA 700 may also be configured to receive data from a detected SDU 100, such as the data collected by the SDU 100 regarding at least one event. For yet other embodiments, the PA 700 devices may operate in conjunction with an additional wireless communication system, such as a WiFi network system, wherein the plurality of PAs 700 are principally responsible for determining physical location within the facility, the wireless communication system being responsible for additional data collection from each SDU 100.

For at least one embodiment, each PA 700 is in communication with a HUB 702, understood and appreciated to be a central collection point for SDU 100 data/events. In varying embodiments, the HUB 702 can be as simple as a network router that relays events from SDUs 100 to the remote computing system 128, and/or external services, or it can contain extra intelligence (software or hardware) and/or proprietary elements for the purpose of aggregating events, performing analytics, and it may even contain an interface for operator interactions. For yet another embodiment the HUB 702 may be integrated as an element of the remote computing system 128.

It is understood and appreciated that for at least one embodiment, various kinematic events are pre-determined and programmed into the HUB 702 (or other appliance) so that specific kinematic events may be identified and distinguished from other kinematic events—i.e., the jarring forces of walking, standing, sitting, bending, dancing, or other “normal” movements as oppose to threshold forces of a fall, impact, slip, impact, etc . . . .

It is understood and appreciated that for at least one embodiment, various temperature events are pre-determined and programmed into the HUB 702 (or other appliance) so that specific temperature events may be identified and distinguished from other temperature events—i.e., the rapid change in temperature due to removal or insertion in the oral cavity, significant changes in body temperature (excluding eating or drinking), etc . . . .

For yet one alternative embodiment, the SDU 100 may be structed and arranged to derive its own location—such as by incorporating a GPS chip as at least one sensor 116. For such an embodiment, the SDU may optionally communicate directly with the HUB 702 to send location information and to send data in response to one or more events—e.g., the passage of time, detection of an event, response to a ping for identification/information, etc . . . .

The HUB 702 and/or external cloud services/systems 704 may be configured to recognize a report from an accelerometer within the SDU 100 that the motion a user 108 has experienced represents a kinematic event above a pre-determined threshold, i.e., a fall or sudden impact. In response to this detection, the HUB 702 is preconfigured to send an alert message to at least one caregiver 706 or other party. This alert may be in the form of test message, a pre-recorded message, SMS, audio alert, or even AI developed message transmitted to a remote computing device 708 in the possession of the caregiver 706, the message providing the receiving party with the relevant information, such as, but not limited to the location and time of the kinematic event, the person involved in the event, the degree of the kinematic event, whether the person has suffered a similar event in the past, whether any other persons have suffered a similar kinematic event in this area, etc. . . . . For at least one embodiment, the HUB 702 may be further augmented to call for remote emergency assistance, such as a transmission to 911 or other emergency response system for medical assistance and/or an ambulance.

It should be further understood and appreciated that normal kinematic and temperature change events such as eating & chewing are pre-established events filtered from treatment as alarm triggers. However, it can and should certainly be noted, that time and location parameters may be added to the events of eating & chewing. Moreover, eating and chewing in a dining location or at a prescribed time may not trigger an alarm, but eating in an unauthorized location or at an unauthorized time may. As such, the SDU 100 may be advantageous in improving treatment with patients experiencing eating disorders.

The HUB 702 and/or external cloud services 704 may be configured to recognize a report from a sensor 116 within the SDU 100 that the temperature a device registers represents a compliance event above a pre-determined threshold, i.e., use of the SDU 100 for an extended period of time. In response to this detection, for at least one embodiment, the HUB 702 is preconfigured to send an alert message to at least one caregiver 706 or other party. This alert may be in the form of test message, a pre-recorded message, SMS, audio alert, or even AI developed message providing the receiving party with the relevant information, such as, but not limited to the location and time of the temperature event, the person involved in the event, the degree of non-compliance, whether the person is experiencing a potential fever, etc. . . . . For at least one embodiment, the HUB 702 may be further augmented to call for remote assistance, such as a transmission to a care provider.

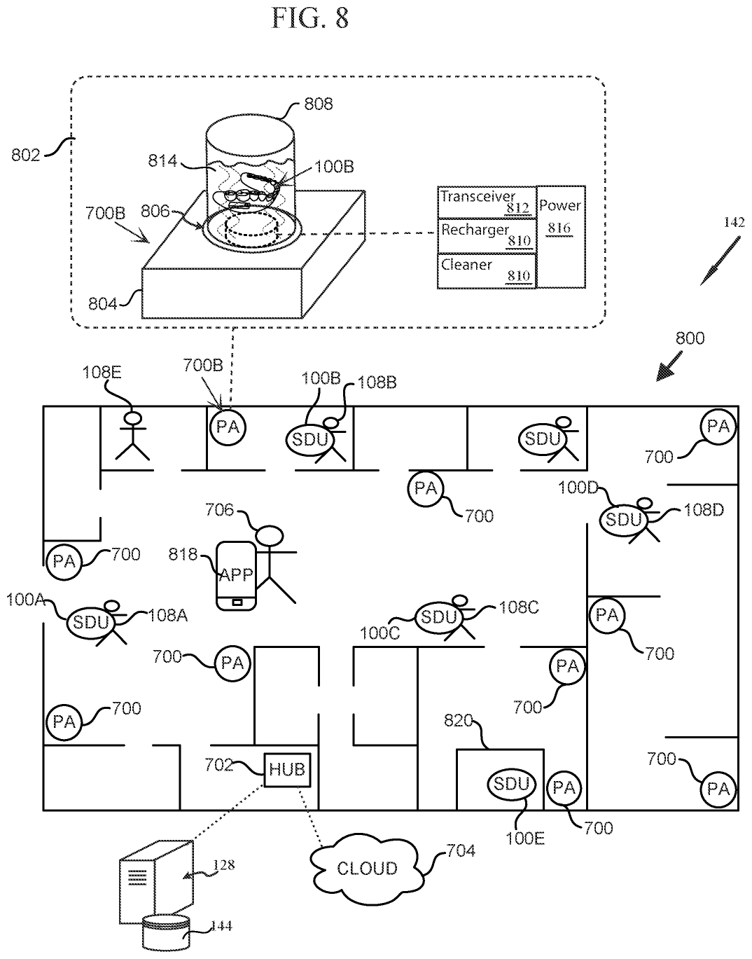

FIG. 8, further expands on FIGS. 1 and 7 to conceptually illustrate an environment for use of at least one embodiment of the present invention. More specifically, there is a facility 800, such as but not limited to a care facility, such as an elder care retreat. As shown, there are a plurality of users 108, such as users 108A, 108B, 108C, 108D and 108E. Each user has a registered SDU 100, correlated as SDU 100A, 100B, 100C, 100D and 100E. For the embodiment shown, a plurality of PAs 700 are shown as well throughout the facility 800.

For at least one embedment, at least some PAs 700 are disposed in door frames or upon/within the lids of trash detectors, exit signs, at all exits, and upon such other common devices as to permit wide, but unobtrusive, disbursement of PAs 700. User 108B has a modified PA 700B that functions also as a cleaning and reharling station for the SDU 100B when removed from the mouth.

As is shown in enlarged section 802, the modified PA 700B in accordance with at least one embodiment, provides a base 804 with a receiving station 806, such as a recessed platform structured and arranged to received an appropriately sized container 808 into which the user or other operator will dispose SDU 100C for nightly cleaning and recharging.

Moreover, when SDU 100B is disposed in the container 808 which is disposed properly in the receiving station 806, the SDU 100B is within the radius of influence for a wireless recharging system 810 that is disposed within the base 804. The base 804 further includes the nearfield wireless transceiver 812 that enables the modified PA 700C to wirelessly communicate and/or detect the proximity of SDU 100B. This wireless transceiver 812, or an additional wireless transceiver 812 may also be optional configured to permit upgrading/reprograming of firmware or software elements within the SDU 100B.

For at least one embodiment, the base 804 also includes a cleaning element 814, such as a sonic agitator, which is disposed proximate to the receiving station 806 so as to induce a bubbling/cleaning action within a cleaning solution disposed in the container 808 along with the SDU 100B. As with other PAs 700, the modified PA 700C is in network communication with the HUB or remote computing system 128.

Base 804 also contains a power supply 814, such as a power converter or other appropriate system which may be connected to a buildings power supply by a power cord, or optionally powered by one or more batteries. In varying embodiments, the modified PA 700B may also be adapted to provide additional features such as an alarm clock, radio, music streaming speaker, digital picture frame, or such other features as may be desired and enjoyed by users 108 of SDU 100 devices. For yet another embodiment, the modified PA 700C may also include an audible and or visual alert which may be configured to remind user 108C of SDU 100C that it is time for SDU 100C to be deposited for cleaning and/or recharging. Further still, the modified PA 700C may also incorporate light detectors and or motion detectors such that modified PA 700C may provide behavioral information, such as whether or not the user is active or sleeping in his or her room.

At least one caregiver 706 is shown within the facility 800, the caregiver 706 having a portable computing device 708 adapted by an application 816 for interaction with the remote computing system 128, and or HUB 702.

In the event that a User 108 should exit the facility 800, the PA 700 disposed proximate to the exit will alert the caregiver 706 in substantially real time. In addition, if a user 108 having an SDU 100 should have an event of significance (a fall, a drop in pulse ox, change in temperature, a kinematic event suggestive of eating outside of prescribed times, etc. . . . ) the caregiver 706 will receive an alert generated by the remote computing system 128 and delivered to his remote computing device 708 by way of the application 816.

FIG. 8 also illustrates that a lost SDU 100E may be advantageously located. More specifically, user 108E has left his or her SDU 100E in room 820. As noted above, for at least one embodiment, the SDU 108B has optionally been equipped with a speaker such that an audible alert may be triggered to assist in locating the SDU 100B should it have fallen behind something, become entangled in clothing or bedding, or otherwise not be easily visible. The general, if not specific location may also be reported to the caregiver 706 by way of the App 818 as a simple text message, e.g., “Bill's SDU shows a present location in room 820.”

With respect to the above discussion of varying embodiments of SDU 100, it will be understood and appreciated that the features and advantages may be provided in yet other embodiments such as an athletic mouth guard, dental splint, night guard, orthodontic retainer, occlusal/bite guards, fluoride trays, etc. . . . which incorporate essentially the same sensor system 106, if not the SDU chip 150. However, it will be understood and appreciated that most of these dental appliances do not provide prosthetic teeth. The compact and sealed nature of the SDU 100 to achieve at least some of the advantages noted above in varying embodiments without external attachments to wires, devices or otherwise rendering the device as obviously distinguishable from a traditional dental appliance (e.g., it appears to the user and feels to the user essentially just the same) will be applicable to users of these alternative embodiments as well for the advantages such as, but certainly not limited to, detection of kinetic events such as falls or impacts, cardiovascular health markers such as blood oxygen level, the user's temperature, loss prevention, and compliance with a prescribed dental care or treatment plan.

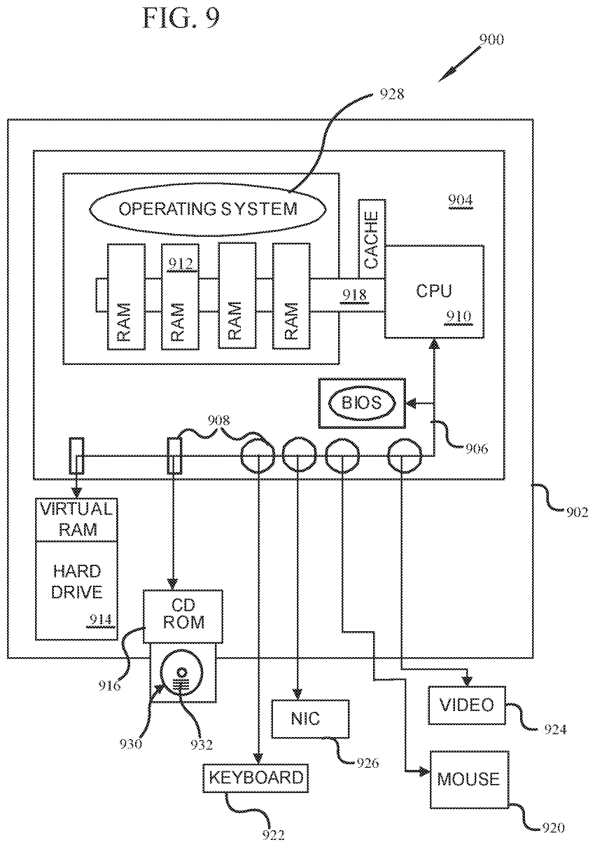

To expand upon the initial suggestion of at least the remote computing system 128, the hub 702, the portable computing device 804 and other systems comprising the environment in which one or more SDUs 100 are deployed, being computer systems adapted to their specific rolls, FIG. 9 is a high level block diagram of an exemplary computer system 900 such as may be provided for one or more of the elements comprising remote computing system 128, the hub 702, the portable computing device 804, and/or other computing devices whether provided as distinct individual systems or integrated together in one or more computer systems.

Computer system 900 has a case 902, enclosing a main board 904. The main board 904 has a system bus 906, connection ports 908, a processing unit, such as Central Processing Unit (CPU) 910 with at least one microprocessor (not shown) and a memory storage device, such as main memory 912, hard drive 914 and CD/DVD ROM drive 916.

Memory bus 918 couples main memory 912 to the CPU 910. A system bus 906 couples the hard disc drive 914, CD/DVD ROM drive 916 and connection ports 908 to the CPU 910. Multiple input devices may be provided, such as, for example, a mouse 920 and keyboard 922. Multiple output devices may also be provided, such as, for example, a video monitor 924 and a printer (not shown). As computer system 900 is intended to be interconnected with other computer systems, a combined input/output device such as at least one network interface card, or NIC 926 is also provided.

Computer system 900 may be a commercially available system, such as a desktop workstation unit provided by IBM, Dell Computers, Gateway, Apple, or other computer system provider. Computer system 900 may also be a networked computer system, wherein memory storage components such as hard drive 914, additional CPUs 910 and output devices such as printers are provided by physically separate computer systems commonly connected in the network.

Those skilled in the art will understand and appreciate that the physical composition of components and component interconnections are comprised by the computer system 900, and select a computer system 900 suitable for one or more of the computer systems incorporated in the formation and operation of a heath care environment utilizing one or more SDUs 100.

When computer system 900 is activated, preferably an operating system 928 will load into main memory 912 as part of the boot strap startup sequence and ready the computer system 900 for operation. At the simplest level, and in the most general sense, the tasks of an operating system fall into specific categories, such as process management, device management (including application and User interface management) and memory management, for example. The form of the computer-readable medium 930 and language of the program 932 are understood to be appropriate for and functionally cooperate with the computer system 900.

Moreover, variations of computer system 900 may be adapted to provide the physical elements of one or more components comprising remote computing system 128, the hub 702, the portable computing device 804, the switches, routers and such other components as may be desired and appropriate for the methods and systems for a smart dental unit (SDU 100) and systems for dental based patient care (SDBP 142) incorporating smart dental units as set forth above.

Changes may be made in the above methods, systems and structures without departing from the scope hereof. It should thus be noted that the matter contained in the above description and/or shown in the accompanying drawings should be interpreted as illustrative and not in a limiting sense. Indeed, many other embodiments are feasible and possible, as will be evident to one of ordinary skill in the art. The claims that follow are not limited by or to the embodiments discussed herein, but are limited solely by their terms and the Doctrine of Equivalents.

What is claimed:

1. A smart dental unit (SDU) restoring one or more teeth, comprising:
a prosthetic base structured and arranged to engage with mouth tissue, the prosthetic base supporting at least one tooth and at least one sensor system, the at least one sensor system including:
at least one unique identifier, structured and arranged to identify the smart dental appliance;
at least one sensor, structured and arranged to detect at least one event;
a processor;
a non-volatile memory coupled to the processor having processor executable instructions to direct the operation of the processor and the at least one sensor for detection of one or more events experienced by the SDU;
a wireless transceiver coupled to the processor and the non-volatile memory and operable to communicate with at least one remote computing system; and
a power source structured and arranged to power at least the processor, the at least one sensor and wireless transceiver; and
wherein in response to the detection of one or more events by the at least one sensor, the processor generates data communicated by the wireless transceiver to the remote computing system.

2. The system of claim 1, wherein the at least one sensor is an accelerometer.

3. The system of claim 1, wherein the at least one sensor is a thermometer.

4. The system of claim 1, wherein the at least one sensor is a pulse oximetry sensor.

5. The system of claim 1, wherein an event is detected at predetermined intervals of time.

6. The system of claim 1, wherein an event is detected when the event exceeds a pre-determined threshold.

7. The system of claim 6, wherein the at least one remote computing system establishes the threshold.

8. The system of claim 7, wherein the threshold is a kinematic event identified as a fall.

9. The system of claim 7, wherein the threshold is a temperature.

10. The system of claim 1, wherein an event is detected in response to a request from the remote computing system.

11. The system of claim 1, wherein the detection of a least one event provides an indication of compliance or noncompliance with a prescribed use of the SDU.

12. The system of claim 1, wherein the prosthetic base is a removable prosthetic appliance.

13. The system of claim 1, wherein SDU further includes an audible alert system, the system structured and arranged to provide an audible alert when activated by a remote computing system.

14. The system of claim 1, wherein the power source is wirelessly rechargeable.

15. A smart dental unit (SDU) restoring one or more teeth, comprising:
a prosthetic base structured and arranged to engage with mouth tissue, the prosthetic base supporting at least one tooth and at least one sensor system, the at least one sensor system including:
at least one unique identifier, structured and arranged to identify the smart dental appliance;
at least one accelerometer;
at least one thermometer;
at least one pulse oximetry sensor;
a processor;
a non-volatile memory coupled to the processor having processor executable instructions to direct the operation at least the processor and the at least one accelerometer and thermometer for detecting a kinetic event and/or present temperature event;
a wireless transceiver coupled to the processor and the non-volatile memory and operable to communicate with at least one remote computing system;
a power source structured and arranged to power at least the processor, accelerometer, thermometer and wireless transceiver; and
wherein in response to the detection of one or more events by the at least one sensor, the processor generating data communicated by the wireless transceiver to the remote computing system.

16. The system of claim 15, wherein an event is detected at predetermined intervals of time.

17. The system of claim 15, wherein an event is detected when the event is exceeds a pre-determined threshold.

18. The system of claim 17, wherein the least one remote computing system establishes the threshold.

19. The system of claim 18, wherein the threshold is a kinematic event identified as a fall.

20. The system of claim 18, wherein the threshold is a temperature.

21. The system of claim 15, wherein an event is detected in response to a request from the remote computing system.

22. The system of claim 15, wherein the detection of a least one event provides an indication of compliance or noncompliance with a prescribed use of the SDU.

23. The system of claim 15, wherein the prosthetic base is a removable prosthetic appliance.

24. The system of claim 15, wherein the SDU further includes an audible alert system, the system structured and arranged to provide an audible alert when activated by a remote computing system.

25. The system of claim 15, wherein the power source power source is wirelessly rechargeable.

26. A method for providing a smart dental unit (SDU) restoring one or more teeth, comprising:
providing a prosthetic base structured and arranged to engage with mouth tissue, the prosthetic base supporting at least one tooth and at least one sensor system, the at least one sensor system comprising:
a sensor disposed within the smart tech repository, the sensor including:
at least one unique identifier, structured and arranged to identify the smart dental appliance;
at least one sensor, structured and arranged to detect at least one event;
a processor;
a non-volatile memory coupled to the processor having processor executable instructions to direct the operation at least the processor and the at least one sensor for detection of one or more events experienced by the SDU;
a wireless transceiver coupled to the processor and the non-volatile memory and operable to communicate with at least one remote computing system;
a power source structured and arranged to power at least the processor, accelerometer, thermometer and wireless transceiver; and
wherein in response to the detection of one or more events by the at least one sensor, the processor generating data communicated by the wireless transceiver to the remote computing system.

27. The method of claim 26, wherein the prosthetic base is a pre-existing prosthetic base adapted to provide the smart tech repository.

28. The method of claim 26, wherein the prosthetic base is fabricated to provide smart tech repository, the at least one sensor disposed within the smart tech repository during the fabrication of the prosthetic base.

29. The method of claim 28, wherein the prosthetic base is at least in part, 3D printed.

30. The method of claim 26, wherein the prosthetic base is a removable prosthetic appliance.

31. The method of claim 26, wherein the prosthetic base further includes at least one mount structured and arranged for attachment to at least one anchor disposed within a person's mouth.

32. The method of claim 28, wherein the at least one sensor is an accelerometer.

33. The method of claim 28, wherein the at least one sensor is a thermometer.

34. The method of claim 28, wherein the at least one sensor is a pulse oximetry system.

35. The method of claim 28, wherein an event is detected at predetermined intervals of time.

36. The method of claim 28, wherein an event is detected when the event is exceeds a pre-determined threshold.

37. The method of claim 36, wherein the least one remote computing system establishes the threshold.

38. The method of claim 36, wherein the threshold is a kinematic event identified as a fall.

39. The method of claim 36, wherein the threshold is a temperature.

40. The method of claim 26, wherein an event is detected in response to a request from the remote computing system.

41. The method of claim 26, wherein the SDU further includes an audible alert system, the system structured and arranged to provide an audible alert when activated by a remote computing system.

42. The method of claim 26, wherein the power system power source is wirelessly rechargeable.
